(19) United States
(12) Patent Application Publication
(10) Pub. No.: US 2014/0373620 A1
(43) Pub. Date: Dec. 25, 2014

(54) Measuring Head

(71) Applicant: Uster Techonologies AG, Uster (CH)

(72) Inventors: Ganapathy Parameswaran, Uster (CH); Martin Kuster, Eschenbach (CH)

(73) Assignee: USTER TECHNOLOGIES AG, Uster (CH)

(21) Appl. No.: 14/383,027

(22) PCT Filed: Jan. 10, 2013
(86) PCT No.: PCT/CH2013/000006

§ 371 Date: Sep. 4, 2014

(30) Foreign Application Priority Data
Mar. 5, 2012 (CH) 290/12

(51) Int. Cl. G01N 33/36

(52) U.S. Cl. CPC G01N 33/365
USPC 73/159

(57) ABSTRACT

A measuring head for testing yarn, having a measuring slit for accommodating the yarn and a first yarn guide element for guiding the yarn through the measuring slit. A second yarn guide element for guiding the yarn outside of the measuring slit. The yarn path outside of the measuring slit is used when at least one of a zero point calibration and an adjustment of the measuring head needs to be performed. A measuring head arranged in this manner is especially suitable for use in classifying yarn defects in the textile laboratory.

### FIELD

This application claims rights and priority on prior pending PCT application PCT/CH2013/000006 filed 2013.01.10 and Swiss patent application 290/12 filed 2012 Mar. 5. The present invention lies in the field of textile quality control. It relates to a measuring head for testing yarn according to the preamble of the first claim. The measuring head is especially suitable for use in classifying yarn defects in the textile laboratory.

### BACKGROUND

A large number of various apparatuses are known for examining or testing yarns. They can be classified according to the application in the two categories of laboratory testing (offline) and testing during the production process (online). The USTER® CLASSIMAT QUANTUM system of the applicant, as described in the brochure “USTER® CLASSIMAT QUANTUM,” Uster Technologies AG, August 2007, is used for the classification of yarn defects such as thick and thin places as well as foreign substances. It is a laboratory device according to its functionality because it is used in the textile laboratory for the detailed examination of samples. The devices of which it consists originate largely from production however. The examined yarn is rewound on a winding position of a manual winding machine and scanned with a measuring head. The yarn parameters measured by the measuring head are statistically evaluated by a personal computer, e.g. classified in a two-dimensional classification diagram. The measuring head can be similar with respect to its relevant features to a yarn clearer measuring head, for example of the type USTER® QUANTUM.

Yarn clearers are used in spinning or bobbin winding machines for ensuring the yarn quality. It is the object of yarn clearing to detect defective places such as thick places, thin places or foreign matter in the yarn, to provide evaluation according to specific quality criteria, and to optionally eliminate said defects. For this purpose a yarn clearer measuring head comprises a measuring slit through which the yarn passes along its longitudinal direction. At least one sensor for scanning the moved yarn is arranged along the measuring slit. Frequently used sensor principles are the capacitive one (e.g. EP-0'924'513 A1) and the optical one (WO-93/13407 A1). The yarn clearer measuring head further contains an electronic circuit for evaluating the sensor signal and for comparing the signal with predetermined quality criteria, e.g. a clearing limit. The mentioned components are housed in a measuring head housing. The measuring head housing comes with the task of protecting the sensitive components from mechanical forces, dirt, humidity, electromagnetic radiation and other external influences. The configuration of a yarn clearer measuring head is described for example in U.S. Pat. No. 6,422,072 B1.

Yarn guide elements, so-called yarn guide eyelets, are attached to the measuring head upstream and downstream with respect to the measuring slit. Their objective is to hold the running yarn in a predefined position in the measuring slit. The yarn guide elements have a substantially V-shaped incision, so that the yarn position is well-defined by the respective apex of the incision.

A measuring head requires timeouts in between for zero point calibration and for adjustment, in which no yarn is situated in the measuring slit. Such timeouts occur repeatedly in normal operation of a yarn clearer on a bobbin winding machine during the removal of a defective place and change of cop. If the measuring head is used in the textile laboratory for classifying yarn defects, the above reasons for timeouts do not occur at all or occur only very rarely. An automatic yarn lifting apparatus can be used in order to remove the yarn from the measuring slit and to reintroduce it into the measuring slit in this type of application, as known from CN-101'706'337 A for example. Such apparatuses are complex and expensive however.

### SUMMARY

It is an object of the present invention to provide a measuring head for testing yarn, which is optimized for the use of classifying yarn defects as described above.

This and other objects are achieved by the measuring head in accordance with the invention for testing yarn as defined in the first claim. Advantageous embodiments are provided in the dependent claims.

The invention is based on the idea of providing guide elements on the measuring head for at least two different yarn paths. The one yarn path extends through the measuring slit and is used for measurement, whereas the other yarn path extends outside of the measuring slit and is used when at least one of a zero point calibration and an adjustment needs to be performed. The yarn can be redirected as required from the one to the other yarn path and back again. The change between the yarn paths occurs manually or automatically. A measuring head arranged in this manner is especially suitable for use in classifying yarn defects in the textile laboratory.

Accordingly, the measuring head in accordance with the invention for testing yarns comprises a measuring slit for accommodating the yarn and a first yarn guide element for guiding the yarn through the measuring slit. It further comprises at least one second yarn guide element for guiding the yarn outside of the measuring slit.

The measuring slit firstly defines a longitudinal direction which coincides with the longitudinal direction of the yarn when the yarn is guided through the measuring slit. It secondly defines a direction of height along which the yarn can be introduced into the measuring slit. It thirdly defines a direction of width which stands perpendicularly to the longitudinal direction and perpendicularly to the direction of height. The first and second yarn guide elements are, in some embodiments, spaced from each other in at least one of the direction of height and the direction of width. In one embodiment, the two yarn guide elements are spaced from each other both in the direction of height and also in the direction of width. In some embodiments, the distance of the two yarn guide elements in the direction of height is between 10 mm and 40 mm, in some embodiments approximately 23 mm, and in some embodiments, the distance of the two yarn guide elements in the direction of width is between 5 mm and 30 mm, in some embodiments approximately 10 mm.

The two yarn guide elements may comprise substantially V-shaped incisions for guiding the yarn. They can consist of a ceramic material. It is especially advantageous if the two yarn guide elements are attached to a common carrier and the carrier is fixed to a housing of the measuring head. The common carrier can be arranged as a plate and the two yarn guide elements can be attached to an edge of the plate. The plate consists for example of a metal, in some embodiments, steel.

In one embodiment, the measuring head comprises a third yarn guide element for guiding the yarn through the measuring slit. The first yarn guide element and the third yarn guide element are attached at the same end of the measuring slit and are spaced from each other in the longitudinal direction. The distance in the longitudinal direction can be between 1 mm and 5 mm, in some embodiments approximately 3 mm. The first yarn guide element and the third yarn guide element are, in some embodiments, attached at the end of the measuring slit which is downstream with respect to an intended yarn running direction.

The measuring head advantageously contains at least one sensor for scanning the yarn, which sensor is arranged along the measuring slit.

### DRAWINGS

The embodiments of the invention are explained below by reference to the drawings, wherein:

FIG. 1 shows a measuring head in accordance with an embodiment of the invention in a perspective view.

FIG. 2 shows a yarn guide plate for the measuring head in accordance with an embodiment of the invention in a top view.

### DESCRIPTION

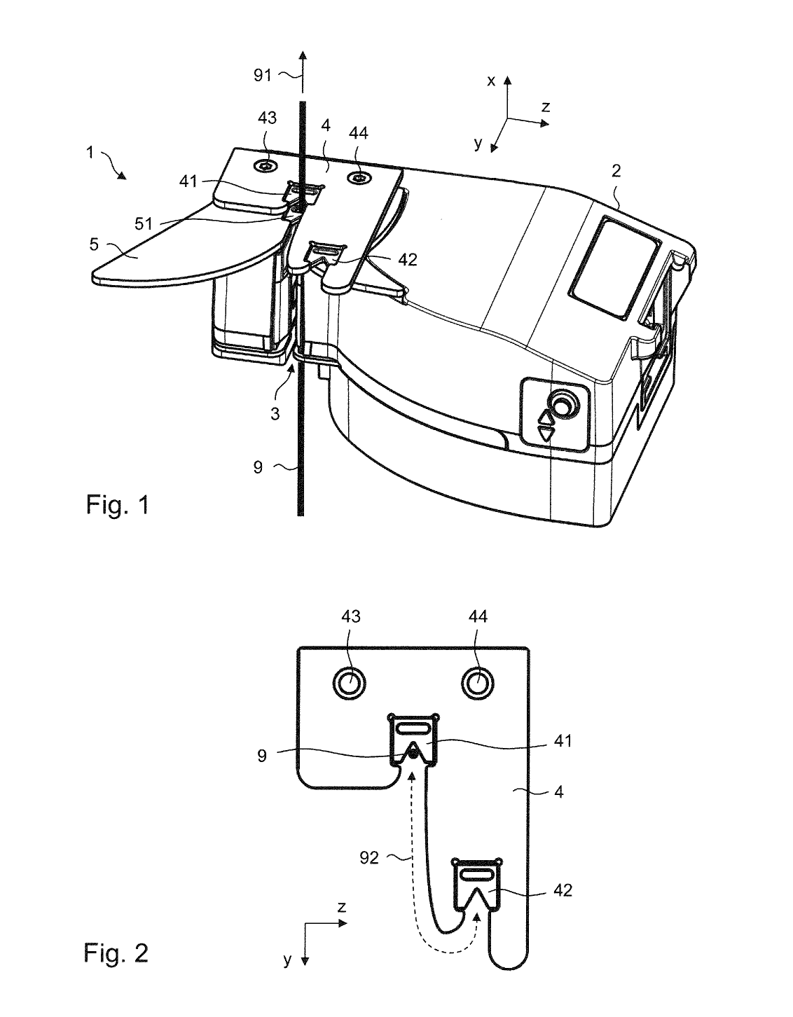

FIG. 1 shows a measuring head 1 in accordance with an embodiment of the invention for testing a yarn 9. It contains a housing 2 with a measuring slit 3 for accommodating the yarn 9. The yarn 9 is moved along its longitudinal direction x through the measuring slit 3, which is indicated by an arrow 91. A sensor (not shown in FIG. 1) for scanning the yarn 9 is arranged in the region of the measuring slit 3 in the housing 2. Electronic evaluation circuits (not shown) are also housed in the housing 2 for evaluating the sensor signal.

A yarn guide plate 4 is fixed to the housing 2 downstream with respect to the measuring slit 3, which yarn guide plate is separately shown in FIG. 2. The yarn guide plate 4 is substantially flat and is disposed in a plane (yz), which is disposed perpendicularly to the longitudinal direction x. It can consist of a metal (e.g. steel) and can have a thickness of 1.5 mm for example. It comprises recesses on its edge for a first yarn guide element 41 and a second yarn guide element 42. The two yarn guide elements 41, 42 comprise substantially V-shaped incisions for guiding the yarn 9. They can consist of a ceramic material and be held by means of glued connections in the recesses of the yarn guide plate 4. The yarn guide plate 4 can be fixed on its part by means of two screwed joints 43, 44 to the housing 2 of the measuring head 1.

The yarn guide plate 4 is configured and arranged on the measuring head 1 in such a way that the first yarn guide element 41 is set up for guiding the yarn 9 through the measuring slit 3, while the second yarn guide element 42 is set up for guiding the yarn 9 outside of the measuring slit 3. The yarn 9 is therefore guided through the first yarn guide element 41 during the measurement and runs through the measuring slit 3. This state is shown in FIGS. 1 and 2. For the purposes of at least one of zero point calibration and adjustment of the measuring head, the yarn 9 is lifted out of the first yarn guiding 41 and the measuring slit 3 and placed in the second yarn guide element 42. This change can occur manually or automatically, wherein the yarn 9 can continue to run and need not be stopped. The yarn is redirected after the change and runs along a different yarn path which does not extend through the measuring slit 3. As long as the yarn 9 is guided through the second yarn guide element 42, the measuring slit 3 is empty and the zero point calibration or adjustment can be performed. The yarn is then lifted out of the second yarn guide element 42 and introduced again into the first yarn guide element 41 and the measuring slit 3. A further measurement can be performed in this state. The reversible change of the yarn 9 from one state to the other is indicated in FIG. 2 by the dashed double arrow 92.

The measuring slit 3 defines a longitudinal direction x which coincides with the longitudinal direction of the yarn 9 when the yarn 9 is guided through the measuring slit 3. The measuring slit 3 further defines a direction of height y, along which the yarn 9 can be introduced into the measuring slit 3. A direction of width z can be defined as the direction which stands perpendicularly to the longitudinal direction x and perpendicularly to the direction of height y. The two yarn guide elements 41, 42 are spaced from each other both in the direction of height y and also in the direction of width z. In one embodiment, the distance of the two yarn guide elements 41, 42 in the direction of height y is between 10 mm and 40 mm, in some embodiments approximately 23 mm, and in one embodiment the distance of the two yarn guide elements 41, 42 in the direction of width z is between 5 mm and 30 mm, in some embodiments approximately 10 mm.

A yarn guide plate 5 can be fixed to the housing 2 of the measuring head 1 downstream with respect to the measuring slit 3. A third yarn guide element 51 can be inserted into the yarn guide plate 5, which yarn guide element is also positioned in such a way that it guides the yarn 9 through the measuring slit 3. It is advantageous in some embodiments to provide several yarn guide elements 51, 41 at the downstream end of the measuring slit 3. After passing the measuring slit 3, the yarn 9 can be wound up on a cross-wound bobbin (not shown), wherein it is moved back and forth in the direction of width z for the even winding of the cross-wound bobbin. In some embodiments this so-called traversing movement is not transferred to the yarn section situated in the measuring slit 3 because this could lead to wrong measurements. Tests have shown that two or more yarn guide elements 51, 41 situated in succession in the yarn running direction x lead to better mechanical isolation than a single yarn guide element 51. The third yarn guide element 51 and the first yarn guide element 41 can be spaced from each other at a distance of between 1 mm and 5 mm, in some embodiments approximately 3 mm, in the longitudinal direction x. The distance can be set by means of suitable spacers. At the other, upstream end of the measuring slit 3, a fourth yarn guide element (not shown in the drawings) can be attached which is also used for guiding the yarn 9 through the measuring slit 3.

It is understood that the present invention is not limited to the embodiments as discussed above. The person skilled in the art will be able to derive further variants with knowledge of the invention which also belong to the subject matter of the present invention.

### LIST OF REFERENCE NUMERALS

1 Measuring head

2 Housing

3 Measuring slit

4 Yarn guide plate

41 First yarn guide element

42 Second yarn guide element

43, 44 Screwed joints

5 Yarn guide plate

51 Third yarn guide element

9 Yarn

91 Running movement of the yarn

92 Change in the state of the yarn

x Longitudinal direction

y Direction of height

z Direction of width

1. A measuring head for testing a yarn, comprising:
a measuring slit for accommodating the yarn,
a first yarn guide element for guiding the yarn through the measuring slit, and
a second yarn guide element for guiding the yarn outside of the measuring slit.

2. The measuring head according to claim 1, wherein the measuring slit defines a longitudinal direction (x) that coincides with the longitudinal direction of the yarn when the yarn is guided through the measuring slit, and the measuring slit defines a direction of height (y), along which the yarn can be introduced into the measuring slit, and wherein the two yarn guide elements are spaced from each other in the direction of height (y).

3. The measuring head according to claim 1, wherein the measuring slit defines a longitudinal direction (x) that coincides with the longitudinal direction of the yarn when the yarn is guided through the measuring slit, and the measuring slit defines a direction of height (y), along which the yarn can be introduced into the measuring slit, and wherein the two yarn guide elements are spaced from each other in a direction of width (z), which direction of width (z) stands perpendicularly to the longitudinal direction (x) and perpendicularly to the direction of height (y).

4. The measuring head according to the claim 2, wherein the two yarn guide elements are spaced from each other both in the direction of height (y) and also in the direction of width (z).

5. The measuring head according to claim 4, wherein the distance of the two yarn guide elements is between 10 mm and 40 mm in the direction of height (y) and the distance of the two yarn guide elements is between 5 mm and 30 mm in the direction of width (z).

6. The measuring head according to claim 1, wherein the two yarn guide elements comprise substantially V-shaped incisions for guiding the yarn.

7. The measuring head according to claim 1, wherein the two yarn guide elements consist of a ceramic material.

8. The measuring head according to claim 1, wherein the two yarn guide elements are attached to a common carrier, and the carrier is fixed to a housing of the measuring head.

9. The measuring head according to claim 8, wherein the common carrier is arranged as a plate and the two yarn guide elements are attached to an edge of the plate.

10. The measuring head according to claim 9, wherein the plate consist of a metal.

11. The measuring head according to claim 10, wherein the two yarn guide elements are arranged as ceramic inserts with a respectively V-shaped incision and the ceramic inserts are inserted into the metal plate.

12. The measuring head according to claim 1, wherein:
the measuring slit defines a longitudinal direction (x) that coincides with the longitudinal direction of the yarn when the yarn is guided through the measuring slit, and
the first yarn guide element and a third yarn guide element are attached at the same end of the measuring slit and are spaced from each other in the longitudinal direction (x).

13. The measuring head according to claim 12, wherein the third yarn guide element and the first yarn guide element are spaced at a distance of between 1 mm and 5 mm in the longitudinal direction (x).

14. The measuring head according to claim 12, wherein the first yarn guide element and the third yarn guide element are attached to an end of the measuring slit that is situated downstream with respect to an intended yarn running direction along the longitudinal direction (x).

15. The measuring head according to claim 1, wherein the measuring head contains at least one sensor for scanning the yarn, which sensor is arranged along the measuring slit.
